(19) United States
(12) Patent Application Publication
(10) Pub. No.: US 2023/0414272 A1
(43) Pub. Date: Dec. 28, 2023

(54) MEDICAL DEVICES

(71) Applicant: Boston Scientific Scimed Inc., Maple Grove, MN (US)

(72) Inventors: Yin XIA, Shanghai (CN); Xinzhe PEI, Shanghai (CN); Longsheng CAI, Shanghai (CN); Minfeng HUA, Shanghai (CN); Yingzhi LU, Shanghai (CN); Xiangqi SU, Shanghai (CN); Jizhe YING, Shanghai (CN)

(73) Assignee: Boston Scientific Scimed Inc., Maple Grove, MN (US)

(21) Appl. No.: 18/252,378

(22) PCT Filed: Dec. 15, 2021
(86) PCT No.: PCT/US2021/063504

§ 371 Date: May 10, 2023

(30) Foreign Application Priority Data
Dec. 31, 2020 (CN) 202011619242.0

(51) Int. Cl. A61B 18/14; A99Z 99/00

(52) U.S. Cl. CPC A61B 18/1485; A61B 18/1482; A99Z 99/00; A61B 2018/1405; A61B 2018/1253

(57) ABSTRACT

A medical device includes a shaft, an end cap, and an electrode. The shaft includes a conductive element. The end cap is coupled to a distal end of the shaft. The electrode is coupled to the distal end of the shaft and passes through the end cap. The electrode includes an electrode shaft and a distal tip, and the electrode is electrically connected to the conductive element. The end cap includes a visualization feature.

### TECHNICAL FIELD

Aspects of this disclosure generally relate to medical devices and related methods. Embodiments of the disclosure relate to medical devices that can treat tissue by delivering electrical energy to or into tissue and/or injecting fluid into and/or under tissue.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat tumors, infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, etc. In particular, such procedures may be carried out by inserting an insertion device into a subject's body through a surgical incision, or via a natural anatomical orifice (e.g., mouth, vagina, or rectum), and performing the procedure or operation at a target site with an auxiliary device inserted through the insertion device.

At times, during a medical procedure, a user may use an auxiliary device that includes a catheter and a distally-extending electrode. The electrode may be energized for purposes of cutting, dissecting, ablating, marking, coagulating, cauterizing, or otherwise treating and/or manipulating tissue. The user may rely on a visualization device at a distal end of the insertion device or at a distal end of another device inserted to or near the target site. With the electrode extending from the distal end of the insertion device, the insertion device may at least partially impair the user's ability to visualize the electrode. As a result, the user may have to maneuver the visualization device closer to the electrode, which may be difficult in a small target site or pose other difficulties.

The devices and methods of this disclosure may rectify one or more of the deficiencies described above or address other aspects of the art.

### SUMMARY

Examples of the disclosure relate to, among other things, medical devices and related methods for treating tissue by delivering electrical energy to the tissue with an electrode, while also visualizing at least a portion of the electrode. Each of the examples disclosed herein may include one or more of the features described in connection with any of the other disclosed examples.

A medical device may include a shaft, an end cap, and an electrode. The shaft may include a conductive element. The end cap may be coupled to a distal end of the shaft. The electrode may be coupled to the distal end of the shaft and may pass through the end cap. The electrode may include an electrode shaft and a distal tip, and the electrode may be electrically connected to the conductive element. The end cap may include a visualization feature.

The medical device may include one or more of the following features. The end cap may be at least partially insulating and may include an opening for the electrode to pass through. The visualization feature may form a blind angle of approximately 30 degrees or less when the distal tip of the electrode is extended approximately 1 mm to approximately 3 mm, such as approximately 1.5 mm, from a distal end face of the end cap. The visualization feature may be formed by a tapered portion of the end cap. The tapered portion of the end cap may include a straight taper. The straight taper may form a blind angle of approximately 25 degrees or less. The tapered portion of the end cap may include a rounded taper. The rounded taper may form a blind angle of approximately 25 degrees of less. The tapered portion of the end cap may include one or more tapered side portions, and optionally one or more partially cylindrical portions. The tapered side portion(s) of the end cap may form a blind angle of approximately 25 degrees on at least one side of the end cap. The tapered portion of the end cap may span approximately 80% of a length of the end cap.

The end cap may be at least partially transparent. The end cap may include a ceramic material, such as artificial sapphire or artificial crystal. The visualization feature may form a blind angle of approximately 21 degrees. A distal portion of the shaft may be at least partially transparent, and the end cap and the distal portion of the shaft may form the visualization feature. The distal portion of the shaft may be approximately 5 mm to approximately 15 mm in length, and the visualization feature may form a blind angle of approximately 11 degrees.

In another aspect, a medical device may include a shaft, an end cap, and an electrode. The shaft may include a conductive element. The end cap may be coupled to a distal end of the shaft. The end cap may include a tapered portion. The electrode may be coupled to a distal end of the shaft, and the electrode may pass through the end cap. The electrode may include an electrode shaft and a distal tip, and the electrode may be electrically connected to the conductive element. When the distal tip of the electrode is extended approximately 1 mm to approximately 3 mm from a distal end face of the end cap, the tapered portion of the end cap may form a blind angle of approximately 20 degrees to approximately 30 degrees.

The medical device may include one or more of the following features. The end cap may be at least partially insulating. The tapered portion of the end cap may include a rounded taper. The tapered portion of the end cap may include one or more tapered side portions and one or more partially cylindrical portions.

In yet another aspect, a medical device may include a shaft, an end cap, and an electrode. The shaft may include a conductive element. The end cap may be coupled to a distal end of the shaft. The end cap may be at least partially transparent. The electrode may be coupled to a distal end of the shaft and may pass through the end cap. The electrode may include an electrode shaft and a distal tip, and the electrode may be electrically connected to the conductive element. When the distal tip of the electrode is extended approximately 1.5 mm from a distal end face of the end cap, the at least partially transparent end cap may form a blind angle of approximately 21 degrees or less.

The medical device may include one or more of the following features. The end cap may be at least partially insulating. The at least partially transparent end cap may include artificial sapphire or artificial crystal. A distal portion of the shaft may be at least partially transparent to form a blind angle of approximately 11 degrees.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this disclosure, illustrate exemplary aspects of the disclosure and together with the description, serve to explain the principles of the disclosure.

FIG. 1A illustrates an exemplary medical device, and FIG. 1B illustrates a cross-sectional view of the medical device with a distal portion of the medical device enlarged, according to aspects of this disclosure.

FIG. 2 illustrates a side view of the distal portion of the medical device of FIGS. 1A and 1B, according to aspects of the disclosure.

FIG. 3 illustrates a side view of a distal portion of another exemplary medical device, according to aspects of the disclosure.

FIGS. 4A-4C illustrate views of a distal portion of another exemplary medical device, according to aspects of the disclosure.

FIG. 5 illustrates a side view of a distal portion of another exemplary medical device, according to aspects of the disclosure.

FIG. 6 illustrates a side view of a distal portion of a further exemplary medical device, according to aspects of the disclosure.

### DETAILED DESCRIPTION

Examples of the disclosure include devices and methods for one or more of: facilitating and improving the efficacy, efficiency, and safety of treating and/or manipulating tissue when, for example, applying electrical energy to tissue with an electrode. For example, aspects of the disclosure may provide a user (e.g., physician, medical technician, or other medical service provider) with a treatment device to deliver energy to a target site with the electrode, while also facilitating visualization of the electrode with a proximally located visualization device. Aspects of the disclosure may provide the user with the ability to apply electrical energy and/or heat to tissue using a medical device having an electrode. Aspects of the disclosure may provide the user with the ability to use a smaller electrode, for example, to maneuver in at the target site, while still visualizing the electrode. Alternatively or additionally, the user may use a larger electrode (e.g., an electrode that extends farther from a distal end of the catheter in order to expose a larger amount of the electrode) in order to better visualize the electrode during the procedure. However, the larger electrode may be more difficult to maneuver, may be more likely to contact tissue inadvertently, etc. Accordingly, aspects of the disclosure may provide the user with the ability to apply electrical energy and/or heat with a reduced likelihood of damaging tissue and/or contacting unintended portions of the tissue. Additionally, aspects of the disclosure may be selectively coupled to an existing medical device to improve the visualization of the electrode. Furthermore, aspects of the disclosure may help the user deliver a distal end of the medical device to the target site. Some aspects of the disclosure may be used in performing an endoscopic, laparoscopic, arthroscopic, gynoscopic, thoracoscopic, cystoscopic, or other type of procedure.

Reference will now be made in detail to examples of the disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

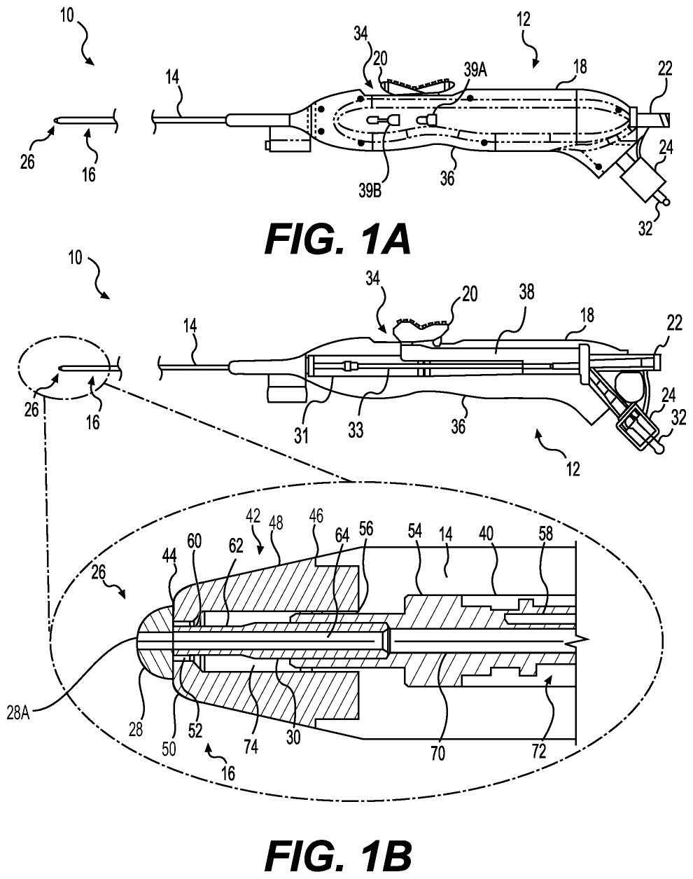

FIGS. 1A and 1B depict a medical device 10 that includes a handle 12 and a shaft 14 having a distal end 16. Handle 12 may include a main body 18 and a movable body 20. Handle 12 also may include a port 22 configured to receive fluid, and a hub 24 configured to receive electrical energy similar to an electrical plug or socket. Distal end 16 includes an end effector, for example, an energy delivery portion or an electrode portion 26 (hereinafter “electrode 26”). Electrode 26 is electrically connected to hub 24, and, as discussed in detail below, may include a channel fluidly connected to, or otherwise in fluid communication with, port 22. Additionally, as shown in FIG. 1B and discussed in detail below, electrode 26 includes a distal tip 28 and an electrode shaft 30. In some aspects, distal tip 28 may be wider (e.g., in a lateral direction away from a longitudinal direction of electrode shaft 30) than electrode shaft 30. For example, distal tip 28 may include a mushroom-like or semi-spherical tip. In some aspects, the size and/or shape of distal tip 28 may help the user to deliver energy and/or treat tissue.

Medical device 10 may be inserted into a body lumen of a subject, either through an insertion device or alone, such that at least a portion of shaft 14 may be within the subject, while handle 12 may remain outside of the subject. Distal end 16 may be positioned at a target site within the subject. From outside of the subject, a user can manipulate handle 12. Additionally, the user may utilize a visualization device (e.g., a camera) positioned at a distal end of an insertion device or another medical device to visualize distal end 16, including electrode 26, at the target site. Movement of movable body 20 relative to main body 18 in a first direction (e.g., the distal direction) may extend electrode 26 relative to shaft 14 (e.g., move electrode 26 distally relative to a distal end of shaft 14). Movement of movable body 20 relative to main body 18 in a second direction (e.g., the proximal direction) may retract electrode 26 relative to shaft 14 (e.g., move electrode 26 proximally relative to a distal end of shaft 14). Although not shown, movable body 20 or additional components of handle 12 may articulate electrode 26 (or electrode 26 and distal end 16) left or right, and/or up or down relative to shaft 14.

In some aspects, handle 12 may be coupled to a fluid source via port 22. Port 22 may be in fluid communication with electrode 26 via an internal lumen 31, which may extend through handle 12 (FIG. 1B) and shaft 14. It is noted that various portions of handle 12 shown in FIG. 1B are not necessarily shown to scale, in order to more fully illustrate various portions of handle 12. In at least one aspect, internal lumen 31 may extend longitudinally through main body 18 of handle 12 and shaft 14 to fluidly connect port 22 to electrode 26. Port 22 may be positioned on a proximal portion of main body 18, for example, a proximal end of main body 18. Alternatively, port 22 may be positioned on a distal or central portion of main body 18. Moreover, port 22 may include a one-way valve, a luer, a seal, threading, and/or any appropriate connection or mating element to help maintain a secure connection between handle 12 and the fluid source, minimize or prevent back-flow (e.g., fluid flowing proximally out of port 22), and/or minimize or prevent leakage. In at least one example, port 22 may include a one-way valve having an outer housing containing an inner elastomeric and/or gelatinous sealing member. In some examples, handle 12 does not include a port 22, e.g., wherein medical device 10 is not used for fluid delivery.

Handle 12 may be coupled to an energy source via hub 24. Hub 24 may include one or more prongs or pins 32 to couple to the energy source. Hub 24 may be electrically coupled to electrode 26 via a conductive element 33, which may be electrically coupled to pin 32 and extend through handle 12 and through at least a portion of shaft 14. The energy source may be, for example, an electrocautery source, a radio frequency generator, a heating source, a current generator, etc. In other aspects, the energy source may be a part of handle 12 (e.g., an internal battery in handle 12).

In at least one aspect, medical device 10 may be used for monopolar electrosurgery, and may include a return electrode positioned remotely from electrode 26 on or otherwise adjacent to the subject. In other aspects, medical device 10 may be used for bipolar electrosurgery. In such instances, electrode 26 may include an active electrode portion, and a return electrode may be provided at or near another portion of electrode 26 and/or shaft 14. In at least one example, two conductive elements may run through shaft 14, where the conductive elements may be electrically isolated from each other, allowing one conductive element to conduct energy to the active electrode and the other conductive element to conduct energy from a return electrode.

Hub 24 may be positioned on main body 18, for example, on a proximal end of main body 18. In at least one aspect, port 22 may extend from the proximal end of main body 18 in a direction parallel to or coaxial with a longitudinal axis of main body 18, and hub 24 may extend from the proximal end of main body 18 at an angle transverse (e.g., approximately 45 degrees) to the longitudinal axis of main body 18. In other aspects, hub 24 may be positioned on a distal or central portion of main body 18, or on movable body 20. According to some examples, main body 18 and/or hub 24 may include a one-way valve, a luer, a seal, threading, and/or any appropriate connection or mating element to help maintain a secure connection between handle 12 and the energy source, minimize or prevent back-flow (e.g., fluid flowing from port 22 and/or internal lumen 31 and proximally out of hub 24), and/or minimize or prevent leakage.

In at least one aspect, as shown in FIG. 1B, pin 32 may extend through hub 24 transverse to a longitudinal axis of handle 12, and may be electrically and physically connected to conductive element 33, such as a wire, a cable, and/or a braided sheath. Conductive element 33 may be electrically conductive or otherwise include an electrically conductive component or portion, and conductive element 33 may extend longitudinally through internal lumen 31 and through shaft 14. As shown in FIG. 1B, fluid delivered through port 22 may surround at least a portion of conductive element 33. In at least one aspect, conductive element 33 may include one or more layers of insulation to help insulate conductive element 33 from the fluid in internal lumen 31. As mentioned above, a second conductive element may be provided as a return pathway when medical device 10 has a bipolar configuration.

As mentioned, handle 12 may control the extension and/or retraction of electrode 26 relative to the distal end 16 of shaft 14. For example, main body 18 may include a slot 34, and movable body 20 may be slidably positioned within slot 34. For example, main body 18 may be configured to be held by a user's hand, and movable body 20 may be configured to be controlled by the movement of the user's thumb. For example, a side of main body 18 opposite to movable body 20 may include one or more contours 36, which may help the user grip main body 18. Movable body 20 may be lockable in one or more positions relative to main body 18, and/or may be spring-biased in a direction (e.g., toward a proximally retracted position).

Movable body 20 may be coupled to a drive element, and the drive element may impart distal and/or proximal movement to at least a portion of electrode 26 based on relative movement between main body 18 and movable body 20. In at least one aspect, the drive element may be physically coupled (directly or indirectly) to movable body 20, such that movement of movable body 20 extends or retracts the drive element, and thus extends or retracts electrode 26. In such aspects, conductive element 33 may electrically connect pin 32 to electrode 26. For example, conductive element 33 may include slack (e.g., be longer than the distance from pin 32 to electrode 26 or another electrical coupling element) in order to help account for any tortuosity shaft 14 may encounter during delivery and/or to help account for the movement (e.g., extension) of electrode 26 or another electrical coupling element.

In some aspects, conductive element 33 may also act as a drive wire, rod, cable, or the like, such that conductive element 33 may impart distal or proximal movement to at least a portion of electrode 26 while also coupling electrode 26 to hub 24, e.g., to the one or more pins 32, to deliver the energy to (and/or from) electrode 26. As shown in FIG. 1B, movable body 20 may be coupled to conductive element 33 via a coupling mechanism, for example, a coupler 38. In at least one aspect, coupler 38 may be physically coupled (directly or indirectly) to movable body 20, and may also be physically coupled (directly or indirectly) to conductive element 33, such that movement of movable body 20 extends and/or retracts conductive element 33, and thus extends and/or retracts electrode 26. It is noted that coupler 38 and/or other components within handle 12 may help maintain the electrical connection between pin 32 and conductive element 33 when conductive element 33, and thus electrode 26, is in the retracted or extended position. Alternatively, in other aspects, coupler 38 and/or other components within handle 12 may be configured to only electrically connect pin 32 and conductive element 33 when conductive element 33, and thus electrode 26, is in the extended position, or an at least partially extended position.

As shown in FIG. 1A, handle 12 may also include one or more indicators, for example, indicators 39A, 39B. In at least one aspect, indicators 39A, 39B may visually indicate to the user the position of electrode 26 relative to shaft 14. The position of indicators 39A, 39B may also correspond with the position of movable body 20. For example, indicator 39A may be positioned on handle 12 at a position corresponding with a retracted position of movable body 20, and may indicate that electrode 26 is retracted relative to shaft 14. Similarly, indicator 39B may be positioned on handle 12 at a position corresponding with an extended position of movable body 20, and may indicate that electrode 26 is extended relative to shaft 14.

As shown in FIGS. 1A and 1B, shaft 14 extends from a distal portion of main body 18 to distal end 16, and may surround at least a portion of electrode 26. Shaft 14 may include a sheath that surrounds at least a portion of one or more lumens (e.g., lumen 31) and a drive wire (e.g., conductive element 33). In other aspects, shaft 14 may be an extrusion that includes one or more lumens extending from handle 12 to distal end 16.

The enlarged portion of FIG. 1B illustrates additional exemplary features of shaft 14 and distal end 16. Electrode 26 includes distal tip 28 and electrode shaft 30. Electrode 26 may be positioned within a portion of an end cap 42 of distal end 16. End cap 42 may be coupled to distal end 16 of shaft 14, and shaft 14 and end cap 42 may partially overlap, as shown in FIG. 1B. End cap 42 may include a distal end face 44. End cap 42 may be at least partially electrically insulating. For example, all or part of end cap 42 may be formed of a ceramic material or another non-conductive material. In some examples, only distal end face 44 and an internal portion of end cap 42 that contacts and/or surrounds electrode 26 may be electrically insulating. Distal end face 44 includes a central opening 52 through which electrode 26 may extend and/or retract. End cap 42 includes a central portion 74 through which electrode shaft 30 may move during the extension and/or retraction. End cap 42 may be fixedly coupled to shaft 14 via welding, an adhesive, crimping, friction fit, or other appropriate coupling material or mechanism.

Electrode 26 may be coupled to a proximal support 54 of distal end 16, which may include an extension 56, which may be cylindrical in shape. Proximal support 54 may be coupled to a portion of the drive wire (e.g., conductive element 33) via a drive wire receiving portion 58, for example, via welding, an adhesive, crimping, friction fit, or any other permanent or temporary coupling material or mechanism. Extension 56 may extend distally and may receive at least a portion of electrode 26. Electrode 26 and extension 56 may be coupled via welding, an adhesive, crimping, friction fit, or other appropriate coupling material or mechanism. In at least one aspect, extension 56 may allow for different electrodes 26 to be removably coupled to distal end 16. Proximal support 54 includes a support lumen and support lumen 70 fluidly connects port 22 to electrode 26, for example, via a lumen (e.g., lumen 31) through shaft 14.

Electrode 26 and proximal support 54 may be movable relative to end cap 42 in response to the relative movement of movable body 20 and main body 18 of handle 12. For example, with movable body 20 in a proximal position relative to main body 18, electrode shaft 30 may be substantially retracted within end cap 42. In the example shown in FIG. 1B, distal tip 28 has a cross-sectional dimension larger than central opening 52, such that only a distal portion of electrode 26 (e.g., distal tip 28) extends distally beyond end cap 42. Then, as movable body 20 is translated distally relative to main body 18, electrode 26 and proximal support 54 translate distally relative to end cap 42 such that a greater portion of electrode 26 (e.g., electrode shaft 30) extends distally beyond end cap 42 through central opening 52.

Alternatively, central opening 52 may be larger than distal tip 28, and with movable body 20 in the proximalmost position, electrode 26 (including distal tip 28) may be fully retracted within central opening 52 of end cap 42. Furthermore, in at least one aspect, movable body 20 may have an equilibrium position relative to main body 18, and the equilibrium position may correspond to electrode shaft 30 being partially extended from end cap 42.

As shown in the enlarged portion of FIG. 1B, electrode shaft 30 includes a distal portion 60 adjacent to distal tip 28 and a longitudinal portion 62. Electrode shaft 30 may include one or more graduated portions, for example, with varying diameters, which may aid in the coupling of electrode shaft 30 to proximal support 54, may help to form one or stop surfaces (e.g., abutting an internal portion of end cap 42, etc.). In some aspects, electrode shaft 30 also may include a lumen 64 extending through electrode shaft 30, for example, extending longitudinally through a central portion of electrode shaft 30. Lumen 64 may be in fluid communication with port 22 via support lumen 70 through proximal support 54. In at least one aspect, an inner sheath 40 may form at least a portion of the fluid connection between lumen 70 and port 22. Additionally, lumen 64 of electrode shaft 30 may be in fluid communication with an outlet 28A to form a channel to deliver fluid from a distal end (e.g., distal tip 28) of electrode 26.

As mentioned above, in some aspects, medical device 10 may be configured to deliver only energy, and not fluid. In such aspects, electrode shaft 30 optionally does not include lumen 64, proximal support 54 optionally does not include support lumen 70, and/or distal tip 28 optionally does not include outlet 28A. Furthermore, in some aspects, electrode 26 may include one or more insulating portions.

Electrode 26 may have a length of approximately 5 mm to approximately mm, for example, approximately 10 mm. Electrode 26, for example, electrode shaft 30, may have a cross-sectional dimension (e.g., diameter) of approximately 0.3 mm to approximately 1.0 mm, for example, approximately 0.4 mm to approximately mm. A largest portion of distal tip 28 (e.g., the proximal end of distal tip 28) may have a diameter of approximately 0.4 mm to approximately 1.5 mm, for example, approximately 0.6 mm to approximately 1.0 mm. These dimensions are exemplary only; distal tip 28 and/or electrode shaft 30 may have different sizes and/or shapes than those above.

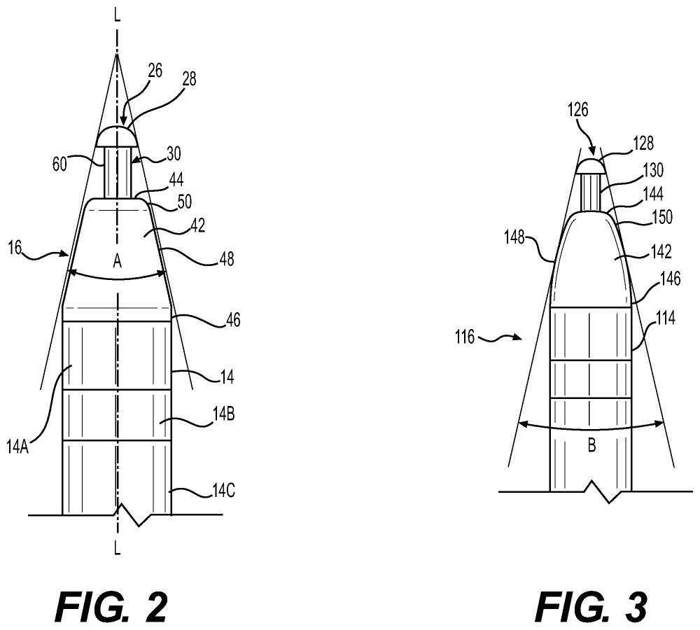

FIG. 2 illustrates additional exemplary aspects of distal end 16 of shaft 14 and electrode 26. As shown in FIG. 2, electrode 26 may be extended relative to end cap 42 and distal end 16 of shaft 14. In the exemplary configuration shown, when electrode 26 is extended, distal tip 28 is spaced away from distal end face 44, and electrode shaft 30 is exposed. For example, electrode 26 may extend from distal end face 44 by approximately 3 mm, approximately 2 mm, approximately 1.5 mm, approximately 1 mm, etc., such as, for example, from approximately 1 mm to approximately 3 mm.

As shown in FIG. 1B and as shown in greater detail in FIG. 2, distal end 16 includes a visualization feature. In this exemplary aspect, the visualization feature is formed by end cap 42, which includes an outer surface that is tapered, for example, such that the radial width and/or cross-sectional dimension (FIG. 1B) reduces from a proximal end of end cap 42 to a distal end of end cap 42. For example, the end cap 42 may have a tapered profile.

As shown in FIGS. 1B and 2, a proximalmost portion 46 of end cap 42 does not taper in this example. As shown, for example, the outermost surface of proximalmost portion 46 may be parallel to the longitudinal axis of distal end 16. Then, as shown, a tapered portion 48 of end cap 42 may extend from proximalmost portion 46 to distal end face 44. Tapered portion 48 may include a straight taper, as shown in the cross-sectional view of FIG. 1B and the side view of FIG. 2. For example, over the span of tapered portion 48, the cross-sectional diameter of end cap 42 may decrease from the proximal portion of tapered portion 48 to the distal portion of tapered portion 48 at a constant rate. In at least one aspect, and as shown in FIGS. 1B and 2, a distalmost portion 50 of end cap 42 may gradually transition (e.g., curve) from tapered portion 48 to distal end face 44.

According to some aspects of the present disclosure, end cap 42 may have a length of approximately 2 mm to 6 mm, for example, approximately 3 mm, and end cap 42 may include a cross-sectional diameter (e.g., as shown in FIG. 1B) of approximately 1.5 mm to approximately 3 mm, for example, approximately 1.8 mm to approximately 2.5 mm. For example, proximalmost portion 46 of end cap 42 may have a cross-sectional diameter of approximately 2.2 mm, and distalmost portion 50 of end cap 42 may have a cross-sectional diameter of approximately 1.4 mm. Distalmost portion 50 of end cap 42 may transition (e.g., curve) from tapered portion 48 to distal end face 44 with a radius of curvature of approximately 0.3 mm. Tapered portion 48 may span a majority (e.g., more than 50% of the length, such as approximately 60% to approximately 98%, e.g., approximately 60%, approximately 70%, approximately 80%, approximately 90%, etc., of the length) of end cap 42. In at least one aspect, tapered portion 48 may include a longitudinal length of approximately 1.8 mm to approximately 2.2 mm, for example, approximately 1.95 mm.

In at least one aspect, in a longitudinal cross-section, tapered portion 48 spans an angle A, for example, approximately 20 degrees to approximately 40 degrees. In at least one aspect, tapered portion spans angle A of approximately 25 degrees. In at least one aspect, tapered portion 48 may extend at an angle of approximately 12.5 degrees relative to longitudinal axis L on each side of longitudinal axis L. In this manner, tapered portion 48 may help to improve the visualization of electrode 26 by not blocking the view of the visualization device (e.g., camera) at a distal end an insertion device or separate device, except for angle A. For example, with electrode 26 extended from end cap 42, the user may be able to visualize electrode 26, as long as the visualization device (e.g., camera), is not within angle A. For example, electrode 26 may be extended approximately 1.5 mm from end cap 42, and tapered portion 48 may allow for increased visualization of electrode 26, including distal tip 28, when treating tissue or maneuvering electrode 26 at the target site. Tapered portion 48 may also help in the delivery of distal end 16 to the target site.

Furthermore, in some aspects, a distal portion of shaft 14 may include one or more sections, e.g., sections 14A, 14B, and 14C. Sections 14A, 14B, and 14C may include different material properties (e.g., different flexibilities or rigidities, different conductivities or insulating properties, etc.). In at least one aspect, one or more sections, for example, section 14B, may be at least partially conductive, such that with electrode 26 in a certain position (e.g., retracted), energy may be delivered through an electrical connection from electrode 26 to section 14B, for example, to help mark tissue at or near the target site. In other aspects, the one or more of sections 14A, 14B, or 14C may include different colors, indications, etc., which may help the user deliver, maneuver, etc. medical device 10 to and/or around the target site.

FIG. 3 illustrates a side view of a shaft 114 and distal end 116 of another exemplary medical device, which may include any of the components of medical device 10 above. Shaft 114 and distal end 116 may be similar to shaft 14 and distal end 16, respectively, with corresponding components. For example, distal end 116 includes an electrode 126 with a distal tip 128 and an electrode shaft 130, and an end cap 142 with a distal end face 144.

Distal end 116 includes a visualization feature formed by end cap 142. In this example, visualization feature is provided by an outer surface of end cap 142 that is tapered, for example, such that the radial width and/or cross-sectional thickness reduces from a proximal end of end cap 142 to a distal end of end cap 142. As shown in FIG. 3, in this example, end cap 142 includes a curved or rounded taper (e.g., rather than a straight taper as illustrated in FIG. 2). As shown in FIG. 3, a proximalmost portion 146 of end cap 142 does not include a taper. For example, the outer surface of proximalmost portion 146 may be parallel to the longitudinal axis of distal end 116. Then, a tapered portion 148 of end cap 142 may extend from proximalmost portion 146 to distal end face 144. Tapered portion 148 may include a curved taper, e.g., a variable rate of taper. For example, the slope of the taper of tapered portion 148 may increase from a proximal portion of tapered portion 148 to a distal portion of tapered portion 146. Moreover, tapered portion 148 may include a curved transition from proximalmost portion 146 to distal end face 144. In at least one aspect, and as shown in FIG. 3, a distalmost portion 150 of end cap 142 may gradually transition (e.g., curve) from tapered portion 148 to distal end face 144.

In at least one aspect, end cap 142 may have a length of approximately 2 mm to 6 mm, and end cap 142 may include a cross-sectional diameter of approximately 1.5 mm to approximately 3 mm, for example, approximately 1.8 mm to approximately 2.5 mm. For example, proximalmost portion 146 may include a cross-sectional diameter of approximately 2.2 mm, and distalmost portion 50 of end cap 42 may have a cross-sectional diameter of approximately 1 mm to approximately 1.4 mm. Tapered portion 148 may span a majority (e.g., more than 50% of the length, such as approximately 60% to approximately 98%, e.g., approximately 60%, approximately 70%, approximately 80%, approximately 90%, etc., of the length) of end cap 142. In at least one aspect, tapered portion 148 may include a longitudinal length of approximately 2 mm to approximately 3 mm, for example, approximately 2.5 mm.

In at least one aspect, in a longitudinal cross-section, tapered portion 148 spans an angle B, for example, approximately 20 degrees to approximately 40 degrees. In at least one aspect, tapered portion 148 spans angle B of approximately degrees. In at least one aspect, tapered portion 148 may extend at an angle of approximately 12.5 degrees relative to longitudinal axis of distal end 116 on each side of the longitudinal axis of distal end 116. In this manner, tapered portion 148 may help to improve the visualization of electrode 126 by not blocking the view of the visualization device (e.g., camera) of an insertion device or separate device, except for angle B. For example, with electrode 126 extended from end cap 142, the user may be able to visualize electrode 126, as long as the visualization device (e.g., camera), is not within angle B. For example, electrode 126 may be extended approximately 1.5 mm from end cap 142, and tapered portion 148 may allow for increased visualization of electrode 126, including distal tip 128, when treating tissue or maneuvering electrode 126 at the target site. Tapered portion 148 may also help in the delivery of distal end 116 to the target site.

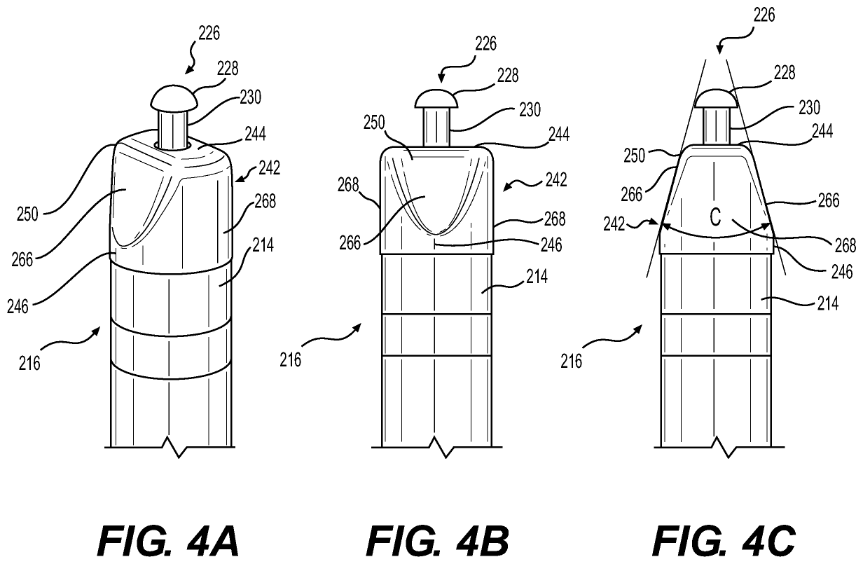

FIGS. 4A-4C illustrate various views of shaft 214 and distal end 216 of another exemplary medical device, which may include any of the components of medical device 10 above. Shaft 214 and distal end 216 may be similar to shaft 14 and distal end 16, respectively, with similar components. For example, distal end 216 includes an electrode 226 with a distal tip 228 and an electrode shaft 230, and an end cap 242 with a distal end face 244. FIG. 4A illustrates a perspective view of distal end 216. FIG. 4B illustrates a side view of distal end 216, and FIG. 4C illustrates another side view of distal end 216.

Distal end 216 includes a visualization feature formed by end cap 242. In this example, the visualization feature is provided by one or more wedge-like or tapered side portions 266 of end cap 242 that are tapered, for example, such that the radial width and/or cross-sectional thickness reduces from a proximal end of end cap 242 to a distal end of end cap 242. End cap 242 also includes one or more rounded non-tapered or partially cylindrical portions 268. For example, as shown in FIGS. 4A-4C, end cap 242 may include two tapered side portions 266. Tapered side portions 266 may form opposite (e.g., circumferentially opposite) sides of end cap 242. Additionally, as shown, end cap 242 may include two cylindrical portions 268 that connect tapered side portions 266. Cylindrical portions 268 of end cap 242 may be substantially the same size (cross-sectional diameter) as shaft 214, and may also include substantially the same circumferential curvature (e.g., partially circular).

In at least one aspect, as shown in FIGS. 4A-4C, tapered side portion(s) 266 may be substantially straight in the longitudinal direction, for example, including a consistent taper from proximalmost portion 246 to distal end face 244 of distalmost portion 250. Alternatively, one or more of tapered side portions 266 may include a curved, or variable taper similar to the curvature shown in FIG. 3. For example, as discussed with respect to tapered portion 146 of FIG. 3, the slope of the taper of tapered side portions 266 may increase from a proximal portion of tapered side portions 266 to a distal portion of tapered side portions 266. Moreover, in some examples, a proximalmost portion 246 of end cap 242 does not include a taper. For example, the outer surface of proximalmost portion 246 may be parallel to the longitudinal axis of distal end 216. In at least one aspect, distalmost portion 250 of end cap 242 may gradually transition (e.g., curve) from tapered side portions 266 to distal end face 244.

In at least one aspect, end cap 242 may have a length of approximately 2 mm to 6 mm, and end cap 242 may have a cross-sectional diameter of approximately 1.5 mm to approximately 3 mm, for example, approximately 1.8 mm to approximately 2.5 mm. For example, proximalmost portion 246 of end cap 242 may have a cross-sectional diameter of approximately 2.2 mm, and distalmost portion 250 of end cap 242 may have a cross-sectional diameter of approximately 0.5 mm to approximately 1.6 mm, for example, approximately 0.8 mm to approximately 1.3 mm. Distalmost portion 250 of end cap 242 may transition (e.g., curve) from tapered portion 248 to distal end face 244 with a radius of curvature of approximately 0.3 mm. Tapered side portions 266 may span a majority (e.g., more than 50%, such as approximately 60% to approximately 98%, e.g., approximately 60%, approximately 70%, approximately 80%, approximately 90%, etc.) of the length of end cap 242. In at least one aspect, tapered side portions 266 may include longitudinal lengths of approximately 2.4 mm to approximately 2.8 mm, for example, approximately 2.6 mm. Moreover, end cap 242 may include one, three, four, or more tapered side portions 266. The one or more tapered side portions 266 may be substantially the same size (e.g., longitudinal length, circumferential width, etc.), or may have different sizes and/or angles as other side portions 266. The one or more tapered side portions 266 may be evenly or unevenly arranged or spaced around the circumference of end cap 242, and optionally may be connected by cylindrical portions 268. In at least one example, end cap 242 includes two or more tapered side portions 266 directly connected to each other, without cylindrical portions 268.

In at least one aspect, in a longitudinal cross-section, two tapered side portions 266 of end cap 242 span an angle C (angle C being formed by the slopes of the two tapered side portions 266), for example, approximately 20 degrees to approximately 40 degrees. In at least one aspect, tapered portions 266 span angle C of approximately 25 degrees. In one aspect, tapered side portions 266 may extend at an angle of approximately 12.5 degrees relative to longitudinal axis of distal end 216 on each side of the longitudinal axis of distal end 216. In this manner, tapered side portions 266 may help to improve the visualization of electrode 226 by not blocking the view of the visualization device (e.g., camera) of an insertion device or separate device, except for angle C. For example, with electrode 226 extended from end cap 242, the user may be able to visualize electrode 226, as long as the visualization device (e.g., camera), is not within angle C. For example, electrode 226 may be extended approximately 1.5 mm from end cap 242, and tapered side portions 266 may allow for increased visualization of electrode 226, including distal tip 228, when treating tissue or maneuvering electrode 226 at the target site. It is noted that tapered side portions 266 may help in visualization of electrode 226, and/or may also help with the delivery of distal end 216 to the target site.

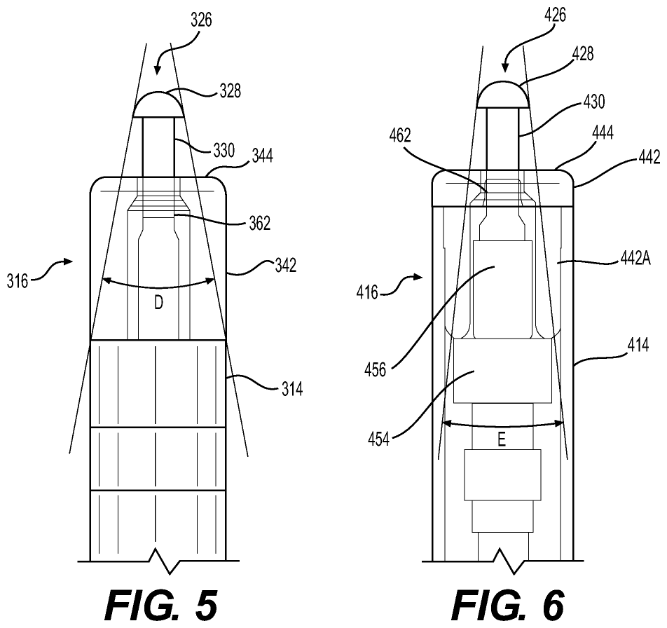

FIG. 5 illustrates a side view of a shaft 314 and distal end 316 of another exemplary medical device, which may include any of the components of medical device 10 above. Shaft 314 and distal end 316 may be similar to shaft 14 and distal end 16, respectively, with similar components. Distal end 316 includes an electrode 326 with a distal tip 328 and an electrode shaft 330, and an end cap 342 with a distal end face 344.

Distal end 316 includes a visualization feature formed by end cap 342. In this example, end cap 342 is at least partially transparent. As shown in FIG. 5, end cap 342 may include a substantially constant cross-sectional dimension (e.g., diameter), and the circumference of end cap 342 may be substantially similar to the circumference of shaft 314. End cap 342 may comprise a transparent material, for example, a ceramic material such as artificial sapphire, artificial crystal, or another material that is at least partially ceramic and at least partially insulating. In some configurations, one or more proximal portions of electrode 326 disposed within end cap 342, for example, longitudinal portion 362, may be at least partially visible through end cap 342.

In at least one aspect, end cap 342 may have a length of approximately 2 mm to 6 mm, and end cap 342 may have a cross-sectional diameter of approximately 1.5 mm to approximately 3 mm, for example, approximately 1.8 mm to approximately 2.5 mm. The exposed portion of end cap 342 may include a length of approximately 2.4 mm to approximately 2.8 mm, for example, approximately 2.6 mm, and a cross-sectional diameter of approximately 2.0 mm to approximately 2.4 mm, for example, approximately 2.2 mm. End cap 342 may transition (e.g., curve) from a side portion to distal end face 344 with a radius of curvature of approximately 0.3 mm. In at least one aspect, with end cap 342 being at least partially transparent, shaft 314 may be the only impediment in visualizing electrode 326, including distal tip 328. In such aspects, shaft 314 may form an angle D relative to the proximal end of distal tip 328.

As shown, because end cap 342 is at least partially transparent in this example, the angle D may be less than an angle formed by outer surfaces of the end cap 342. For example, the angle D may be approximately 15 degrees to approximately 40 degrees. In at least one aspect, angle D is approximately 18 degrees to approximately 25 degrees, for example, approximately 21 degrees. In at least one aspect, the distal end of a side of shaft 314 (e.g., in a longitudinal cross-section) and a side of distal tip 328 may form an angle (corresponding to half the angle D) of approximately 10.5 degrees. For example, as shown in FIG. 5, each side of the distal end of shaft 314 and respective side of distal tip 328 of electrode 326 may extend at angle of approximately 10.5 degrees relative to the longitudinal axis of distal end 316. In this aspect, because end cap 342 is at least partially transparent, distal end 316 may include an angle D of approximately 21 degrees. Accordingly, end cap 342 may help to improve the visualization of electrode 326 by not blocking the view of the visualization device (e.g., camera) of an insertion device or separate device, except for angle D. For example, with electrode 326 extended from end cap 342, the user may be able to visualize electrode 326, as long as the visualization device (e.g., camera), is not within angle D. In at least one aspect, electrode 326 may be extended approximately 1.5 mm from end cap 342, and transparent end cap 342 may allow for increased visualization of electrode 326, including distal tip 328, when treating tissue or maneuvering electrode 326 at the target site.

FIG. 6 illustrates a side view of a shaft 414 and distal end 416 of another exemplary medical device, which may include any of the components of medical device 10 above. Shaft 414 and distal end 416 may be similar to shaft 14 and distal end 16, respectively, with corresponding components. For example, distal end 416 includes an electrode 426 with a distal tip 428 and an electrode shaft 430, and an end cap 442 with a distal end face 444.

Distal end 416 includes a visualization feature formed by end cap 442 and at least a portion (e.g., a distal portion) of shaft 414, which are at least partially transparent. As shown in FIG. 6, end cap 442 may include a substantially constant cross-sectional dimension (e.g., diameter), and the circumference of end cap 442 may be substantially similar to the circumference of shaft 414. Additionally, in some aspects, end cap 442 may include an exposed portion with a shorter longitudinal length than other exemplary end caps discussed herein (e.g., end cap 42, 142, 242, or 342). As shown in FIG. 6, end cap 442 may include a proximal extension 442A, and a portion of shaft 414 (e.g., a portion of distal end 416) may overlay at least a portion of proximal extension 442A. End cap 442 may comprise a transparent material, for example, a ceramic material such as artificial sapphire, artificial crystal, or another material that is at least partially transparent and at least partially insulating.

Additionally, at least a distal portion of shaft 414 (e.g., distal end 416) may comprise, for example, a material that is at least partially transparent and at least partially insulating. In at least one aspect, approximately 5 mm to approximately 15 mm, for example, approximately 10 mm, of the distalmost portion of shaft 414 (e.g., distal end 416) may comprise the at least partially transparent and at least partially insulating material. In at least one aspect, the distalmost portion of shaft 414 may be formed of polypropylene (PP), polytetrafluoroethylene (PTFE), polyvinyl chloride (PVC), polyethylene terephthalate (PET), polycarbonate (PC), etc. In some aspects, the at least partially transparent and at least partially insulating material that forms at least the distal portion of shaft 414 may be the same material that forms end cap 442. In other aspects, the at least partially transparent and at least partially insulating material that forms at least the distal portion of shaft 414 may be a different material than the material the forms end cap 442. For example, the distal portion (e.g., distal end 416) of shaft 414 may be at least partially deflectable and insulating. In these aspects, in some configurations, proximal portions of electrode 426, for example, longitudinal portion 462, and/or proximal support elements, for example, electrode proximal support 454 and electrode cylindrical extension 456, may be at least partially visible through end cap 442 and/or shaft 414.

In at least one aspect, end cap 442 may have a length of approximately 1 mm to 4 mm, and end cap 442 may have a cross-sectional diameter of approximately 1.5 mm to approximately 3 mm, for example, approximately 1.8 mm to approximately 2.5 mm. An exposed portion of end cap 442 may include a length of approximately 0.4 mm to approximately 0.8 mm, for example, approximately 0.6 mm, and the exposed portion of end cap 442 may include a cross-sectional diameter of approximately 2.2 mm. End cap 442 may transition (e.g., curve) from a side portion to distal end face 444 with a radius of curvature of approximately 0.3 mm. The at least partially transparent portion of shaft 414 may have a length of approximately 5 mm to approximately 15 mm, for example, approximately 10 mm.

In at least one aspect, with end cap 442 and a distal portion of shaft 414 being at least partially transparent, proximal support elements, for example, electrode proximal support 454 and electrode cylindrical extension 456, may be the only impediment in visualizing electrode 426, including distal tip 428. In this aspect, the proximal support elements may form an angle E relative to the proximal end of distal tip 428. As shown, electrode proximal support 454 may be the widest component of electrode 426 that is not transparent. Because end cap 442 and the distal portion of shaft 414 are at least partially transparent, electrode proximal support 454 may form angle E of approximately 5 degrees to approximately 20 degrees. In at least one aspect, angle E is approximately 8 degrees to approximately 15 degrees, for example, approximately 11 degrees. In one aspect, the distal end of each side (e.g., in a longitudinal cross-section) of electrode proximal support 454 may form an angle that is half of angle E, e.g., an angle of approximately 5.5 degrees. For example, as shown in FIG. 6, each side of the distal end of electrode proximal support 454 and the respective side of distal tip 428 of electrode 426 may extend at angle of approximately 5.5 degrees relative to the longitudinal axis of distal end 416. In this aspect, because end cap 442 and the distal portion of shaft 414 are at least partially transparent, distal end 416 may include an angle of approximately 11 degrees.

Accordingly, end cap 442 and shaft 414 may help to improve the visualization of electrode 426 by not blocking the view of the visualization device (e.g., camera) of an insertion device or separate device, except for angle E. For example, with electrode 426 extended from end cap 442, the user may be able to visualize electrode 426, as long as the visualization device (e.g., camera), is not within angle E. In at least one aspect, electrode 426 may be extended approximately 1.5 mm from end cap 442, and end cap 442 and the distal portion of shaft 414 may allow for increased visualization of electrode 426, including distal tip 428, when treating tissue or maneuvering electrode 426 at the target site.

The various electrodes discussed herein are capable of modifying physical properties of tissue when in contact with tissue by delivering energy (e.g., radio frequency energy). As discussed above, the energy delivered may be monopolar or bipolar energy. The various electrodes may be coupled to a shaft, with the shaft configured to extend into a body lumen or cavity of a subject. The shaft includes an electrical element traversing the shaft and connecting the electrode to an energy source, for example, in the handle or coupled to the handle. Additionally various aspects of this disclosure (e.g., rounded distal tip 28, end caps 42, 142, 242, 342, 442, etc.) may help to form an atraumatic tip on the distal end of the medical device as the medical device is delivered to the target site.

As discussed, the electrodes may also be coupled to an actuation member (e.g., movable body 20), for example, in the handle or coupled to the handle, that allows a user to translate the electrode relative to the shaft. The electrode may be translatable between at least a first position in which a cutting shaft (e.g., longitudinal portion 62), of the electrode is retracted within the shaft, and a second position in which the cutting shaft is extended beyond the shaft and exposed. In both the first and second positions, the distal portion (e.g., distal tip 28) may be extended and exposed beyond the shaft, and not retracted within the shaft. Moreover, the handle may allow for the electrodes to be positioned in one or more intermediate position (i.e., a position in which only a portion of electrode shaft 30 is exposed).

For example, with the electrode in an extended position to treat the target site, the user may utilize a visualization device (e.g., a camera) that is positioned proximal to the distal end of the medical device, for example, on a separate insertion device, catheter, etc. The various visualization features discussed herein may improve the ability of the user to visualize the electrode with the visualization device. For example, the various visualization features discussed herein may reduce the angle formed by the distal end of the medical device (e.g., the blind angle). As a result, the electrode may be extended from the distal end of the medical device, and, as long as the visualization device is not within the blind angle formed by one or more elements at the distal end of the medical device, the electrode will be within the field of view of the visualization device. Moreover, with an increased field of view, the visualization device may be positioned farther away from the electrode, and the electrode need not be extended far from the distal end of the medical device. With the electrode extending a shorter distance from the medical device, the user may apply electrical energy or heat to tissue at the target site, with a reduced likelihood of damaging tissue or contacting unintended portions of the tissue.

While principles of the disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

1-15. (canceled)

16. A medical device, comprising:
a shaft including a conductive element;
an end cap coupled to a distal end of the shaft, wherein the end cap is at least partially insulating and includes an opening; and
an electrode coupled to the distal end of the shaft and passing through the opening in the end cap,
wherein the electrode includes an electrode shaft and a distal tip,
wherein the electrode is electrically connected to the conductive element, and
wherein the end cap includes a visualization feature.

17. The medical device of claim 16, wherein the visualization feature forms a blind angle of approximately 30 degrees or less when the distal tip of the electrode is extended approximately 1 mm to approximately 3 mm from a distal end face of the end cap.

18. The medical device of claim 17, wherein the visualization feature is formed by a tapered portion of the end cap.

19. The medical device of claim 18, wherein the tapered portion of the end cap includes a straight taper.

20. The medical device of claim 19, wherein the straight taper forms a blind angle of approximately 25 degrees or less.

21. The medical device of claim 18, wherein the tapered portion of the end cap includes a rounded taper.

22. The medical device of claim 21, wherein the rounded taper forms a blind angle of approximately 25 degrees of less.

23. The medical device of claim 18, wherein the tapered portion of the end cap includes one or more tapered side portions, and one or more partially cylindrical portions.

24. The medical device of claim 23, wherein the one or more tapered side portions of the end cap forms a blind angle of approximately 25 degrees on at least one side of the end cap.

25. The medical device of claim 24, wherein the tapered portion of the end cap spans approximately 80% of a length of the end cap.

26. The medical device of claim 16, wherein the end cap is at least partially transparent.

27. The medical device of claim 26, wherein the end cap comprises a ceramic material.

28. The medical device of claim 27, wherein the visualization feature forms a blind angle of approximately 21 degrees.

29. The medical device of claim 28, wherein a distal portion of the shaft is at least partially transparent, and wherein the end cap and the distal portion of the shaft form the visualization feature.

30. The medical device of claim 29, wherein the distal portion of the shaft is approximately 5 mm to approximately 15 mm in length, and wherein the visualization feature forms a blind angle of approximately 11 degrees.

31. A medical device, comprising:
a shaft including a conductive element;
an end cap coupled to a distal end of the shaft, wherein the end cap is at least partially insulating, and wherein the end cap includes a tapered portion; and
an electrode coupled to a distal end of the shaft and passing through the end cap, wherein the electrode includes an electrode shaft and a distal tip, and wherein the electrode is electrically connected to the conductive element,
wherein, when the distal tip of the electrode is extended approximately 1 mm to approximately 3 mm from a distal end face of the end cap, the tapered portion of the end cap forms a blind angle of approximately 20 degrees to approximately 30 degrees.

32. The medical device of claim 31, wherein the tapered portion of the end cap includes a rounded taper.

33. The medical device of claim 31, wherein the tapered portion of the end cap includes one or more tapered side portions and one or more partially cylindrical portions.

34. A medical device, comprising:
a shaft including a conductive element;
an end cap coupled to a distal end of the shaft, wherein the end cap is at least partially insulating, and wherein the end cap is at least partially transparent; and
an electrode coupled to a distal end of the shaft and passing through the end cap, wherein the electrode includes an electrode shaft and a distal tip, and wherein the electrode is electrically connected to the conductive element,
wherein, when the distal tip of the electrode is extended approximately 1.5 mm from a distal end face of the end cap, the at least partially transparent end cap forms a blind angle of approximately 21 degrees or less.

35. The medical device of claim 34, wherein the at least partially transparent end cap comprises artificial sapphire or artificial crystal, and wherein a distal portion of the shaft is at least partially transparent to form a blind angle of approximately 11 degrees.
